(19) United States
(12) Patent Application Publication
(10) Pub. No.: US 2005/0234458 A1
(43) Pub. Date: Oct. 20, 2005

(54) Expanded stabilization of bones

(76) Inventor: Randall J. Huebner, Beaverton, OR (US)

Correspondence Address: KOLISCH HARTWELL, P.C., 520 S.W. YAMHILL STREET, SUITE 200, PORTLAND, OR, 97204, US

(21) Appl. No.: 11/109,985

(22) Filed: Apr. 19, 2005

Related U.S. Application Data
(60) Provisional application No. 60/563,767, filed on Apr. 19, 2004.

(51) Int. Cl. A61B 17/58

(52) U.S. Cl. 606/69

(57) ABSTRACT

Systems, including methods, apparatus, and kits, for expanded stabilization of bones.

### CROSS-REFERENCE TO PRIORITY APPLICATION

This application is based upon and claims the benefit under 35 U.S.C. § 119(e) of the following U.S. provisional patent application, which is incorporated herein by reference in its entirety for all purposes: Ser. No. 60/563,767, filed Apr. 19, 2004.

### CROSS-REFERENCES TO RELATED APPLICATIONS

This application incorporates herein by reference the following U.S. patent applications: Ser. No. 10/716,719, filed Nov. 19, 2003; Ser. No. 10/717,015, filed Nov. 19, 2003; Ser. No. 10/717,399, filed Nov. 19, 2003; Ser. No. 10/717,401, filed Nov. 19, 2003; Ser. No. 10/717,402, filed Nov. 19, 2003; Ser. No. 10/731,173, filed Dec. 8, 2003; Ser. No. 10/873,522, filed Jun. 21, 2004; Ser. No. 10/968,850, filed Oct. 18, 2004; and Ser. No. 11/071,050, filed Feb. 28, 2005.

This application also incorporates herein by reference the following U.S. provisional patent applications: Ser. No. 60/563,860, filed Apr. 19, 2004.

### INTRODUCTION

The human skeleton is composed of 206 individual bones that perform a variety of important functions, including support, movement, protection, storage of minerals, and formation of blood cells. To ensure that the skeleton retains its ability to perform these functions, and to reduce pain and disfigurement, bones that become damaged should be repaired promptly and properly. Typically, a fractured or cut bone is treated using a fixation device, which reinforces the bone and keeps it aligned during healing. Fixation devices may include external fixation devices (such as casts and/or fixators) and/or internal fixation devices (such as bone plates, nails, and/or bone screws), among others.

Bone plates are sturdy internal devices, usually made of metal, that mount directly to the bone adjacent a fracture (or other bone discontinuity). To use a bone plate to repair a bone discontinuity, a surgeon typically (1) selects an appropriate plate, (2) reduces the discontinuity (e.g., sets the fracture), and (3) fastens the plate to bone fragments disposed on opposite sides of the discontinuity using suitable fasteners, such as screws and/or wires, so that the bone plate spans the discontinuity and the bone fragments are fixed in position.

Bone plates may be pre-shaped to fit onto a population-averaged contour of a bone, and onto a region of the bone at which fractures typically occur. These pre-shaped bone plates thus may not extend to regions of the bone that show more variation in the population and/or that represent less common, secondary fracture sites in multiply fractured bones (bones broken into several fragments). For example, bone plates shaped to span more proximal fractures of the distal radius may not have a large enough footprint to secure distal fragments created by additional, more distal fractures of the radius.

### SUMMARY

The present teachings provide systems, including methods, apparatus, and kits, for expanded stabilization of bones.

### BRIEF DESCRIPTION OF THE DRAWINGS

FIG. 1 is a fragmentary view of the bones of the right arm including a multiply fractured distal portion of a radius bone.

FIG. 2 is a plan view of an exemplary bone plate secured to a volar surface region of the fractured radius bone of FIG. 1, in accordance with aspects of the present teachings.

FIG. 3 is a sectional view of the bone plate and radius bone of FIG. 2, taken generally along line 3-3 of FIG. 2, in accordance with aspects of the present teachings.

FIG. 4 is a plan view of an exemplary system for expanded stabilization of a distal portion of a radius bone, including the bone plate of FIG. 2 and an exemplary extension member secured to the bone plate and positioned to stabilize a distal fragment of the radius, in accordance with aspects of the present teachings.

FIG. 5 is a sectional view of the bone plate and radius of FIG. 4, taken generally along line 5-5 of FIG. 4, in accordance with aspects of the present teachings.

FIG. 6 is a plan view of the extension member of FIG. 4 in an adjustable configuration that permits movement of the extension member relative to the bone plate, in accordance with aspects of the present teachings.

FIG. 7 is a sectional view of another exemplary extension member, taken generally as in FIG. 5, in accordance with aspects of the present teachings.

FIG. 8 is a fragmentary sectional view of an exemplary set of plate components that may be included in a kit for expanded stabilization of a fractured distal radius, in accordance with aspects of the present teachings.

FIG. 9 is a fragmentary sectional view of another exemplary set of plate components that may be included in a kit for expanded stabilization of a fractured distal radius, in accordance with aspects of the present teachings.

FIG. 10 is a sectional view of another exemplary system for expanded stabilization of a distal portion of a radius bone, taken generally as in FIG. 5, in accordance with aspects of the present teachings.

FIG. 11 is a view of an exemplary guide device mounted on the bone plate of FIG. 2 adjacent the outer surface of the bone plate, in accordance with aspects of the present teachings.

### DETAILED DESCRIPTION

The present teachings provide systems, including methods, apparatus, and kits, for expanded stabilization of bones. The systems generally include a bone plate for a bone, and an extension member, including, for example, a plate member and/or a wire(s), coupled to the bone plate. The extension member also may engage and/or may be coupled to a region of the bone spaced from the footprint of the bone plate on bone, for example, distal to the distal end of the bone plate, to expand the stabilization capability of the bone plate. In some examples, the extension member may be configured to stabilize a fragment of the distal radius created by an intra-articular fracture, such as a fracture including the lunate facet of the radius. In some examples, the extension member may be coupled alternatively to the bone plate in adjustable and fixed configurations. The adjustable configuration may permit the extension member (and/or a plate member thereof) to move translationally and/or to pivot relative to the bone plate.

The extension member may include a plate member and one or more projections configured to extend from the plate member into bone. The plate member may include an overlapping region that abuts the bone plate and an extension region that extends beyond the perimeter and/or the distal end of the bone plate, to expand the footprint of the bone plate on bone. The plate member, including the overlapping and/or extension region, may bend generally outward, that is, away from the central axis of the bone, to generally follow a corresponding contour of the bone plate and/or bone. The extension region may include one or more openings configured to receive fasteners (such as screws or pins, among others) that extend into bone from an inner surface of the plate member, and/or the extension region may be unitary with projections (such as tines, among others) extending from its inner surface.

The systems of the present teachings may offer a number of advantages for stabilization of bones, such as a distal portion of the radius bone (the distal radius). These advantages may include, for example, (1) expandable stabilization capabilities (e.g., a bone plate with or without an extension member and/or with the extension member in different positions), (2) better stabilization of one or more fragments disposed at least partially (or substantially or entirely) outside the bone plate's footprint on bone, (3) a more customized fit to individual fractured bones, and/or (4) greater adjustability, among others.

Further aspects of the present teachings are described in the following sections, including (I) overview of an exemplary stabilization system; (II) bone plates, (III) extension members, (IV) kits for bone stabilization, (V) application of stabilization systems, and (VI) examples.

### I. OVERVIEW OF AN EXEMPLARY STABILIZATION SYSTEM

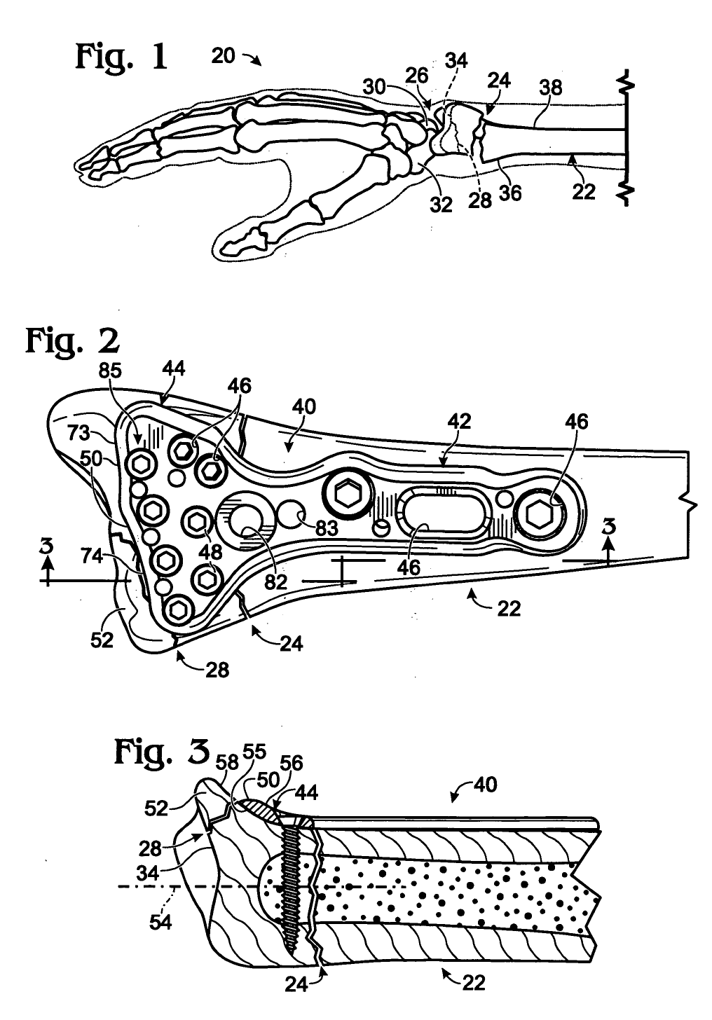

FIG. 1 shows a lateral view of the bones of a right arm 20 exhibiting a multiply fractured distal portion of a radius bone 22. Radius 22 has an exemplary fracture pattern that may be suitable for stabilization (and/or fixation) by the systems of the present teachings. In particular, radius 22 includes an extra-articular fracture 24, spaced from wrist joint 26, and an intra-articular fracture 28 that extends to a facet (articulation surface) of the wrist joint. The wrist joint is defined by articulation between the radial facet (the smooth, concave end of the radius) and adjacent articulation surfaces of lunate 30 and scaphoid 32 carpal bones, disposed medially and laterally, respectively, in the wrist. In the present illustration, intra-articular fracture 28 is disposed medially, that is, extending to lunate facet 34 (the portion of the radial facet that articulates with the lunate bone). A bone plate of the present teachings may be secured adjacent any suitable surface, for example, volar surface 36 or dorsal surface 38 of the radius bone.

FIG. 2 shows an exemplary bone plate 40 secured to the volar surface of fractured distal radius 22. Bone plate 40 may include a proximal (or body) portion 42 secured to the radius, generally proximal to extra-articular fracture 24, and a distal (or head) portion 44 secured distal to extra-articular fracture 24. Each of the proximal and distal portions of the bone plate may define openings 46 (locking (e.g., threaded) or nonlocking) for receiving fasteners, such as bone screws 48 or wires, among others, that secure the bone plate to bone. However, the bone plate may have a distal end or distal perimeter 50 that does not extend far enough distally on the radius to engage and/or fix a distal fragment 52 defined by intra-articular fracture 28.

FIG. 3 shows bone plate 40 and radius 22 in sectional view. The bone plate may be contoured to fit onto the radius, for example, bending outward (upward in the present view), away from a central long axis 54 of the radius, in distal portion 44 of the bone plate. Accordingly, the distal portion (and particularly an inner surface 55 and/or an outer surface 56 of the bone plate in the distal portion) may have a nonplanar shape that corresponds substantially to the anatomical contour of the radius. A contour may be created in the bone plate, for example, during its manufacture, according to the average shape of the radial surface within the population or a suitable subset thereof.

The shape of the radius near its end, for example, as defined by volar incline region 58, towards the medial side of the radius, may exhibit considerable variability within the population. In particular, within the population, the volar incline region generally extends at different angles relative to the long axis of the radius and may have different sizes. This variability in contour and size may preclude design of a pre-shaped bone plate that extends far enough to stabilize radius fragments defined by intra-articular fractures, particularly fractures that include lunate facet 34 (and/or the scaphoid facet and/or radial styloid) of the radius, such as distal fragment 52 created by intra-articular fracture 28.

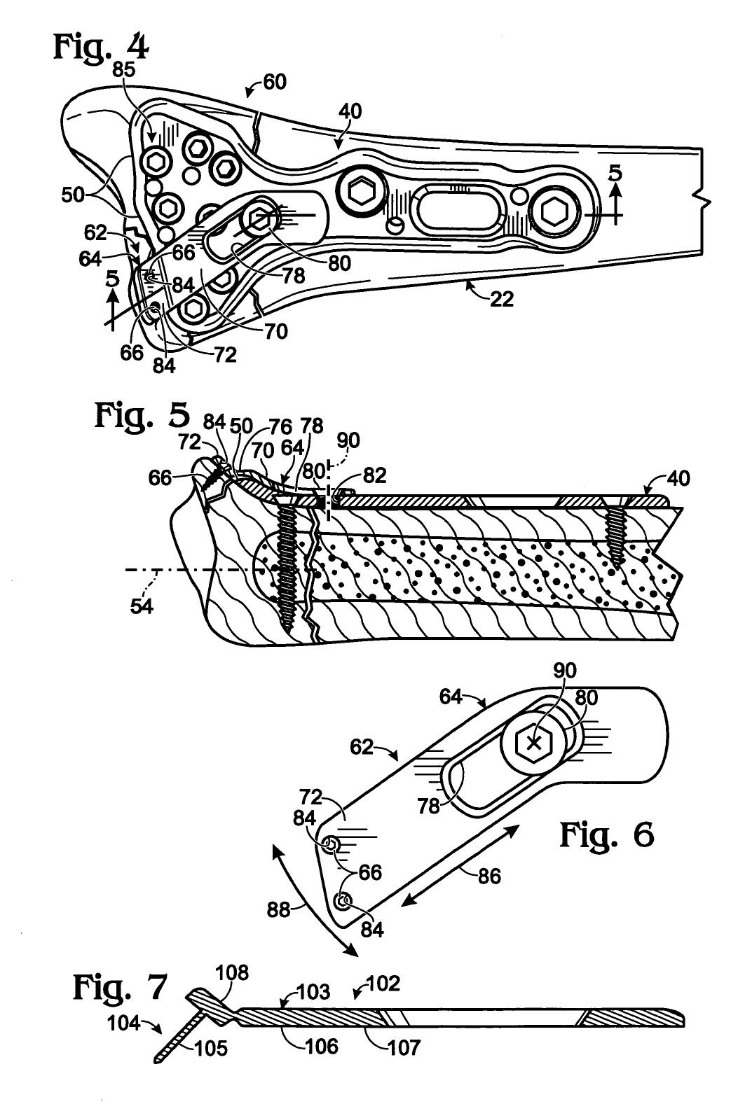

FIGS. 4 and 5 show an exemplary system 60 for expanded (and/or expandable) stabilization of fractured distal radius 22. The system may include a bone plate, such as bone plate 40, and an extension member 62 coupled to the bone plate. The extension member may include a plate member 64 and one or more projections 66 extending from an inner surface of the plate member into bone in a spaced relation to the bone plate (i.e., outside of the footprint of the bone plate on the radius bone). Here, the projections are provided by threaded fasteners, such as bone screws. Alternatively, or in the addition, the projections may include and/or be provided by nonthreaded pins, tines, posts, etc. The projections and the plate member may be unitary (one piece) or may be provided by distinct components. When distinct components, the projections may lock to the plate member, for example, by threadable coupling, or may not lock to the plate member.

The plate member of the extension member may include an overlapping region 70 and an extension region 72. In some examples, the overlapping region may be larger than the extension region, so that the overlapping region is a major portion and the extension region is a minor portion of the plate member (based, for example, on the area of bone and/or bone plate covered and/or apposed by each portion). The overlapping region of the plate member may configured to be disposed generally inside the perimeter of the bone plate, so that the overlapping region extends along and abuts a face of the bone plate, that is, the outer or inner surface of the bone plate. The extension region may be configured to be disposed generally outside the perimeter of the bone plate, for example, extending adjacent a medial region 73 (or lateral region 74) (see FIG. 2) of distal end 50 and distally beyond the medial (and/or lateral) region of the distal end. The extension region thus may extend to a position adjacent a generally medially (and/or laterally) disposed bone surface region outside and distal to the footprint of the bone plate on bone. The extension region may be at least substantially or completely external to bone (or may extend partially into the bone). The extension region may be parallel or nonparallel to the overlapping region, for example, extending (along a linear, curved, and/or curvilinear path) away (and/or towards) a plane defined by the overlapping region of the plate portion and/or away from central long axis 54 of the radius (see FIG. 5).

The plate member further may include a junction region 76 disposed between the overlapping and extension regions (see FIG. 5). The junction region may be configured to facilitate bending the plate portion, before and/or during application, to change the angular disposition of the overlapping and extension regions. For example, the junction region may have a reduced cross-sectional dimension (e.g., reduced thickness and/or width) and/or may be treated differently (e.g., annealed) relative to flanking regions of the plate member.

The plate member may include one or a plurality of openings configured to receive fasteners. Here, the overlapping region of the plate member includes an elongate opening 78, such as an oval slot, configured to receive at least one threaded fastener 80 that couples the plate extension to bone plate 40. In some examples, threaded fastener 80 may be received threadably in a locking (e.g., threaded) aperture 82 of the bone plate (see FIG. 5). Threaded aperture 82 (and adjacent aperture 83) (see FIG. 2) also may be used to couple a guide device to the bone plate (see FIG. 11). The guide device may be used to define guide paths for placement of fasteners through the openings of the bone plate and into bone (or through bone and into one or more openings of the plate in a retrograde direction). The plate member thus may replace the guide device after the bone plate is secured to bone, allowing the same aperture(s) of the bone plate to be used for distinct purposes during installation of the bone plate and extension member. The plate member also may include one or more openings 84 configured to be positioned generally outside of the perimeter of the bone plate. These openings may be configured to receive fasteners that engage and stabilize one or more distal bone fragments.

The plate member may extend any suitable distance proximally along the bone plate. For example, the plate member may extend to a position proximal to a row or set 85 of two or more bone plate openings (see FIGS. 2 and 4) disposed adjacent the distal end (or distal perimeter) of the bone plate. The bone plate openings may be configured to receive any suitable fasteners, such as bone screws, and may be locking or nonlocking. The plate member may overlap and/or completely cover one or more of the openings of the row, or may extend in a nonoverlapping relationship through the row or around an end of the row. In some examples, the plate member may extend proximally at least to an aperture disposed proximal to the row or set of openings, and may couple to the bone plate using the aperture (such as aperture 82 (see FIGS. 2 and 5)). The aperture may be disposed in the head portion or body portion of the bone plate, or generally between these portions. In some examples, the plate member (or aperture) may extend (or may be disposed) proximally at least about halfway from the distal end to the body portion. In some examples, the plate member may extend to a central portion of the bone plate, and/or the aperture may be disposed in the central portion. In any case, the plate member may gain a number of advantages by extending farther proximally, including (1) a greater overlap and engagement with the bone plate and thus greater stability, (2) a longer radial arm for pivotal movement and thus greater potential for medial-lateral adjustment, (3) no occupancy of distal openings, so that each distal opening may receive a bone screw for bone fixation, and/or (4) better use of a central portion of the plate in which bone screw placement into bone may be less critical or unnecessary for fixation, among others.

FIG. 6 shows plate member 64 of extension member 62 in an adjustable configuration. In particular, the plate member may be coupled to the bone plate using threaded fastener 80, but the fastener may not be tightened fully against plate member 64. Accordingly, the plate member may move translationally, parallel to the long axis of elongate opening 78, shown at 86, to adjust the proximal-distal position of the plate member (and the size of extension region 72). Alternatively, or in addition, the plate extension may pivot, shown at 88, about an axis 90 defined by fastener 80, to adjust the medial-lateral position of extension region 72. Adjustment of the position of the plate member relative to the bone plate (and/or coupling of the plate member to the bone plate) may be performed before, during, or after the plate member is coupled to and/or engaged with a distal bone fragment. Accordingly, fasteners 66 may be placed into openings 84 of the plate member and into bone before, during, and/or after adjustment of the position of the plate member. The plate member may be fixed in position by tightening fastener 80 against the plate member.

### II. BONE PLATES

Bone plates of the present teachings generally comprise any plate-like fixation device configured for attachment to bone. The plates may be of a sturdy yet malleable construction. Generally, the plates should be stiffer and stronger than the section of bone spanned by each plate, yet flexible (e.g., springy) enough not to strain the bone significantly. The plates may be unitary, that is, formed as one piece, or may include two or more discrete components. The two or more discrete components may be connected through a mechanical joint that enables translational and/or pivotal movement to adjust the shape and/or size of the bone plates. Further aspects of unitary and multi-component bone plates that are adjustable are described in the patent applications listed above under Cross-References, which are incorporated herein by reference, particularly U.S. patent application Ser. No. 10/716,719, filed Nov. 19, 2003; U.S. patent application Ser. No. 10/717,015, filed Nov. 19, 2003; U.S. patent application Ser. No. 717,399, filed Nov. 19, 2003; and U.S. patent application Ser. No. 10/717,402, filed Nov. 19, 2003.

A. Plate Shade and Structure

The bone plates of the present teachings may have any shape suitable for use on their intended target bones. The bone plates may be shaped for use on any suitable bone or bones, including a bone of the arms (such as a humerus, a radius, an ulna, etc.), a bone of the legs (such as a femur, a tibia, a fibula, etc.), or the like. The bone may have any suitable condition to be treated such as a fracture, a malunion, a nonunion, a cut (an osteotomy), a structural weakness, an undesirable length and/or angulation, and/or the like. The condition may affect any suitable portion of the bone, such as a diaphyseal (shaft) and/or a metaphyseal (end) region of the bone. In exemplary embodiments, the condition affects a distal portion of a radius bone.

The bone plates may have any suitable contour. In some examples, the bone plates may be supplied in a precontoured configuration (e.g., by pre-operative bending and/or machining, among others) to include an inner surface that is complementary to an external surface region of a target bone, such as the distal radius (e.g., a distal volar, distal dorsal, distal lateral, and/or distal medial surface). The bone plates thus may be precontoured according to an average or representative surface geometry of a bone. Alternatively, or in addition, the bone plates may be contoured peri-operatively (e.g., by bending), to adjust their shape before and/or during their installation on bone, to improve, for example, the fit of the bone plates on a target bone for particular individuals.

The bone plates may be configured for use on the distal radius. The distal radius, as used herein, refers to any portion of the radius bone that is spaced from the proximal end of the radius bone. Generally, the distal radius refers to a distal portion that is less than about one-half or one-third the length of the radius bone. The bone plates of the present teachings may be configured preferably to fix radius bones having fractures or other discontinuities disposed in the distal about one-fourth of the radius, although they may be used more generally to repair any suitable fracture.

Each bone plate may be configured for use on any suitable side or sides of the body. For example, the bone plate may be configured for use on both the left radius and the right radius, such as when the bone plate has bi-lateral mirror symmetry. Alternatively, each bone plate may be configured for use on either the left radius bone or the right radius bone, but not both.

The bone plates may include a proximal portion and a distal portion configured to have a corresponding relative disposition on the distal radius. The proximal portion thus may be configured to be disposed substantially proximal to a bone discontinuity, and the distal portion may be configured to be disposed substantially distal to a bone discontinuity, so that these portions are attached to the radius adjacent opposing sides of the discontinuity.

The proximal and distal portions may be connected to one another through a bridge or junction region of each plate. The junction region may be joined unitarily to each of the proximal and distal portions, to provide a plate member of unitary construction, or may provide a site at which discrete proximal and distal plate components are connected to each other, to provide a plate member of non-unitary construction. The junction region may be configured to allow proximal and distal portions of each plate to slide, bend, turn, and/or twist relative to one another. Alternatively, or in addition, the junction region may provide a site at which a guide device and/or an extension member may be attached to the plate. Fasteners also or alternatively may be placed into bone from the junction region. However, the junction region may span a discontinuity in bone so that fastener placement into bone from the junction region may be less desirable than in other portions of the plate.

The bone plates may have any suitable shape defined by the perimeter of the plate. In some examples, the proximal portion may be generally linear, and the distal portion may widen relative to the proximal portion. For example, the plates may be generally T-shaped, with an axially disposed proximal portion and a transversely disposed distal portion, and/or may have a widened fan-like head (the distal portion) connected to an elongate stem or body (the proximal portion). The width of the proximal portion may be generally constant or more vary along its length. Furthermore, the edges of the proximal portion may be generally linear, curved, and/or sinuous. For example, the width may vary according to the lateral disposition of one or more apertures that are offset and/or staggered in disposition, to produce one or more lateral bulges in the perimeter.

The bone plates may be configured to reduce irritation to the bone and surrounding tissue. For example, the plates may have a low and/or feathered profile to reduce their protrusion into adjacent tissue and rounded, burr-free surfaces to reduce the effects of such protrusion.

The plates may be generally elongate (at least before bending), with a length L, a width W, and a thickness T. Here, length L>width W>thickness T. In use, the long axis of the plates, and particularly of the proximal portion, may be aligned with the long axis of the radius bone and/or may extend obliquely and/or transversely relative to the long axis.

The thickness of the plates generally is defined by a distance between inner (bone-facing) and outer (bone-opposing) surfaces of the plates. The thickness of the plates may vary according to the intended use, for example, to make the plates thinner as they extends over protrusions (such as processes, condyles, tuberosities, and/or the like), reducing their profile and/or rigidity, among others. The thickness of the plates also may be varied to facilitate use, for example, to make the plates thinner, to facilitate bending where they typically need to be contoured peri-operatively. In this way, the plates may be thicker and thus stronger in regions where they typically do not need to be contoured, for example, regions of the plates that are placed along the shaft of the bone, among others. In some examples, the proximal portion of each bone plate may be thicker than the distal portion and/or the bridge region disposed between the proximal and distal portions. A thinner bridge region may permit adjustment of the relative angular disposition of the proximal and distal portions by bending and/or twisting the plate at the bridge region. A thinner distal portion may reduce irritation by reducing the profile of this portion of the plate. In some examples, the proximal and distal portions may have about the same thickness, or the distal portion may be thicker than the proximal portion.

B. Plate Apertures

The plates generally include a plurality of apertures (openings) configured to perform similar or different functions. The apertures may be adapted to receive fasteners for affixing the plates to the bone. Alternatively, or in addition, the apertures may be configured to alter the local rigidity of the plate and/or to facilitate blood flow to a fracture or surgical site to promote healing, among others. In some examples, one or more apertures of a plate may be configured for coupling a guide device to the bone plate. Each aperture may have any suitable shape, including non-elongate (such as circular) or elongate (such as oval, elliptical, rectangular, etc.). Apertures may be formed and/or tapped (threaded) pre-operatively, such as during the manufacture of the plates, and/or peri-operatively, such as with the plates disposed on bone. Further aspects of tapping apertures peri-operatively are included in U.S. patent application Ser. No. 10/873,410, filed Jun. 21, 2004, which is incorporated herein by reference.

Individual apertures may be locking or nonlocking. Exemplary locking apertures include a thread, ridge, and/or lip for engaging complementary structure on a fastener, to restrict axial movement of the fastener into or out of the aperture. The thread and/or the wall of the aperture may be configured to stop over-advancement of a fastener. For example, the thread may terminate in a dead end adjacent the inner surface of the plate, and/or the thread or aperture may taper inward toward the inner surface. Alternatively, or in addition, structure to stop over-advancement of the fastener may be included in the fastener. Other locking apertures are described in the patent applications listed above under Cross-References, which are incorporated herein by reference, particularly U.S. patent application Ser. No. 11/071,050, filed Feb. 28, 2005.

The bone plates may have one or more openings configured as slots. A slot is any opening having a length that is greater than its width. The slot may be linear, arcuate, or angled, among others. The slot may include a counterbore structure to receive a head of a bone screw. The counterbore structure may be configured, as in a compression slot, to exert a force generally parallel to the long axis of the slot when a bone screw is advanced against the counterbore structure. Slots may extend axially, that is, in general alignment with the long axis of the plate, or transversely, that is, substantially nonparallel to the long axis, that is, oblique to the long axis or orthogonal to the long axis. Each bone plate may have one or more axial slots and one or more transverse slots. The slots may be used to adjust the translational and/or angular disposition of each bone plate on bone. Further aspects of slots that may be included in the bone plates of the present teachings are described further in the patent applications listed above under Cross-References, which are incorporated herein by reference, particularly, U.S. patent application Ser. No. 10/717,015, filed Nov. 19, 2003.

The bone plates may be configured to receive wires. Each bone plate thus may include one or more holes (generally of smaller diameter) extending through the plate between inner and outer surfaces of the plate. Alternatively, or in addition, the bone plates may be configured to receive and retain wires that extend over (or under) the plates, rather than through the plates, from bone spaced from the plates. Further aspects of bone plates configured to secure wires are described in the patent applications listed above under Cross-References, which are incorporated herein by reference, particularly U.S. Provisional Patent Application Ser. No. 60/563,767, filed Apr. 19, 2004.

C. Plate Materials

A bone plate of the present teachings may be at least substantially formed of, or may include, any suitable biocompatible material(s) and/or bioresorbable material(s). Exemplary biocompatible materials that may be suitable for the bone plate include (1) metals/metal alloys (for example, titanium or titanium alloys, alloys with cobalt and chromium (such as cobalt-chrome), stainless steel, etc.); (2) plastics (for example, ultra-high molecular weight polyethylene (UHMWPE), polymethylmethacrylate (PMMA), polytetrafluoroethylene (PTFE), polyetheretherketone (PEEK), and/or PMMA/polyhydroxyethylmethacrylate (PHEMA)); (3) ceramics (for example, alumina, beryllia, calcium phosphate, and/or zirconia, among others); (4) composites (for example, carbon-fiber composites); (5) bioresorbable (bioabsorbable) materials or polymers (for example, polymers of α-hydroxy carboxylic acids (e.g., polylactic acid (such as PLLA, PDLLA, and/or PDLA), polyglycolic acid, lactide/glycolide copolymers, etc.), polydioxanones, polycaprolactones, polytrimethylene carbonate, polyethylene oxide, poly-β-hydroxybutyrate, poly-β-hydroxypropionate, poly-δ-valerolactone, poly(hydroxyalkanoate)s of the PHB-PHV class, other bioresorbable polyesters, and/or natural polymers (such as collagen or other polypeptides, polysaccharides (e.g., starch, cellulose, and/or chitosan), any copolymers thereof, etc.); and/or the like. In some examples, one or more of these materials may form the body of a bone plate and/or a coating thereon.

Further aspects of bone plates that may be suitable for use in the bone plates of the present teachings are described in the patent applications listed above under Cross-References, which are incorporated herein by reference, particularly U.S. patent application Ser. No. 10/716,719, filed Nov. 19, 2003; U.S. patent application Ser. No. 10/717,015, filed Nov. 19, 2003; U.S. patent application Ser. No. 717,399, filed Nov. 19, 2003; U.S. patent application Ser. No. 10/717,402, filed Nov. 19, 2003; and U.S. patent application Ser. No. 10/731,173, filed Dec. 8, 2003.

### III. EXTENSION MEMBERS

The systems of the present teaching may include and/or use extension members that couple to bone plates and extend beyond the perimeter of the bone plates.

The extension members may have any suitable shape. The extension members may be precontoured to fit onto a bone plate and/or may be contoured (e.g., bent) peri-operatively to fit onto the plate. The extension members may be elongate, with lengths greater than their widths, or the lengths and widths may be about the same. In some examples, the extension members (and/or plate members thereof) may be generally plate-like, having a length and a width that are substantially greater than the thickness of the extension members. Alternatively, the extension members (and/or wire members thereof) may be generally wire-like, having cross-sectional dimensions that are similar and substantially less than the length. Further aspects of extension members including wires are described in the following patent application, which is incorporated herein by reference: U.S. Provisional Patent Application Ser. No. 60/563,767, filed Apr. 19, 2004.

The extension members (and/or plate members) may have any suitable dimensions, including any suitable length, width, and thickness. The length of an extension member may be, for example, greater than, about the same as, or less than the length of a bone plate for which the extension member is configured. In some examples, the extension member may be less than about one-half of the length of the bone plate, and/or may be greater than about one-tenth or about one-fifth the length of the bone plate. The width of the extension member may be substantially less than the width of the bone plate, particularly a distal (or head) portion of the bone plate. In some examples, the extension member may have a width that is less than about one-half the width of the bone plate, and/or the width may be greater than about one-tenth the width of the bone plate and/or greater than the width of at least one locking aperture in the bone plate. The width of the extension member may be constant or may vary along the long axis of the extension member. In some examples, the extension member may narrow (or widen) in an extension and/or bridge region of the extension member. The thickness of the extension member may be greater than, about the same as, or less than the thickness of the bone plate. In some examples, the thickness of the extension member may be substantially less than the thickness of the bone plate, such as less than about one-half the thickness. The thickness may be constant or may vary, such as decreasing (or increasing) in an extension or bridge region of the extension member.

The extension members (and/or plate members) may include any suitable number of apertures (openings) and/or projections. Each aperture may be circular or noncircular (e.g., oval) and may be locking (e.g., threaded) or nonlocking. Each aperture may include or lack a counterbore disposed toward an outer (or inner) surface of the extension member. One or more apertures may be disposed in each of the overlapping and extension regions, only in the overlapping region, or only in the extension region. The apertures may be sizes to receive a screw, a wire, a pin, and/or the like. The projections may be unitary with plate members or may be separate fastener members. Each projection(s) may extend from any suitable surface of a plate member, such as an inner surface, an outer surface, a distal end, a proximal end, or one or both edges, among others. One or more apertures and/or projections of each extension member may be used in coupling the extension member to a bone plate (such as by receiving a fastener or post).

The extension members may be formed of, or may include, any suitable material. Exemplary materials that may be suitable are described above in Section II for bone plates. The extension members may be formed of the same material as the bone plates, (e.g., metal such as titanium, titanium alloy, or stainless steel, among others) or may be formed of a different material.

### IV. KITS FOR BONE STABILIZATION

The systems of the present teachings may provide kits for stabilizing bones. The kits may include one or more bone plates, one or more extension members and/or plate members for coupling to the bone plates, fasteners (such as bone screws, wires, or the like) for securing the bone plate(s) and/or extension member(s) to bone and/or each other, a guide device, a drill(s), one or more clamps, instructions for use, and/or the like. Some or all of the components of each kit may be provided in a sterile condition, such as packaged in a sterile container.

In some examples, the kits may include a set of two or more extension members (and/or plate members). The extension/plate members may differ in contour (such as the angle between overlapping and extension regions), size (such as overall length and/or length of their extension regions), handedness (such as plate members for use on left and right bone plates), site of plate attachment, thickness, and/or the like.

### V. APPLICATION OF STABILIZATION SYSTEMS

The stabilization systems of the present teachings may provide methods of stabilizing (and/or fixing) bones. The methods may include any combination of the following steps, performed in any suitable order, and any suitable number of times, including once or more than once: (1) select a bone to be stabilized, (2) select a bone plate for the bone, (3) select an extension member for the bone plate (and/or bone), (4) dispose the bone plate on the bone, (5) secure the bone plate to the bone, (6) couple the extension member to the bone plate, and (7) engage the bone with the extension member. Further aspects of the steps are described below.

A bone to be stabilized may be selected. The bone may have any suitable discontinuity, including a fracture, a cut (e.g., produced by an osteotomy), a malunion, a nonunion, etc. The fracture may be a single break or a plurality of connected or separate breaks. In some examples, the fracture may include an extra-articular fracture, an intra-articular fracture, or a combination of these fractures. The bone may be a long bone or another bone of the skeleton. In some examples, the bone is a radius bone, particularly a radius bone that has sustained a fracture, or two or more fractures to a distal section of the bone. In some examples, the radius bone may have sustained an intra-articular fracture that extends to the articular end of the bone, such as to the lunate facet of the bone. The bone discontinuity may be reduced, e.g., the fracture set. Reduction may be performed before and/or after the bone plate and extension member are applied to the bone. Selecting a bone to be stabilized also may include creating an incision through soft tissue on the volar, dorsal, lateral, and/or medial side of the bone, to access the bone. This and other suitable steps of the methods may be performed under sterile conditions and/or in a sterile field, for example, during surgery in an operating room.

A bone plate for the bone may be selected. The bone plate may be selected according to the bone to be stabilized and thus may have a size and shape corresponding to the bone. For example, the bone plate may be contoured so that its inner surface fits on the exterior of the bone. In some examples, the bone plate may be pre-contoured (e.g., by bending, machining, and/or casting, among others) according to an average anatomy of a bone within a population. The bone plate may be configured for use on both sides of the skeleton, or may be configured for use on a right bone or a left bone, but not both. In some examples, the bone plate may include indicia (e.g., one or more alphanumeric characters, one or more words, a color, a bar code, etc.) to identify the bone plate, the bone for which the bone plate is configured, the size of the bone plate, the handedness of the bone plate, and/or the like.

An extension member for the bone plate may be selected. The extension member (and/or a plate member thereof) may be selected from a set of two or more extension members of different sizes, shapes, contours, handedness, etc. Accordingly, the extension member (and/or plate member) may include indicia, as described above for bone plates, to facilitate selection of a suitable extension member. The extension member may be shaped to fit onto the bone plate and thus may be selected according to its ability to fit onto the bone plate, the position and/or number of a bone fragment(s) (e.g., medial, lateral, distal, volar, dorsal, and/or the like) to be stabilized, the position of a bone discontinuity that creates the fragment(s), etc. The extension member (and/or plate member) may be shaped peri-operatively (for example, bent before and/or during a surgery in which the bone plate is installed) and/or may be pre-shaped, e.g., during manufacture.

The bone plate may be disposed on the bone. The bone plate may be disposed on any suitable surface along and/or around the bone, such as on a distal volar surface of a radius bone, among others. The bone plate may be disposed such that the bone plate spans a fracture in the bone, such as an extra-articular fracture.

The bone plate may be secured to the bone. The bone plate may be secured by placement of fasteners through openings of the bone plate. The fasteners may include wires and/or bone screws, among others. The fasteners may be placed through bone plate openings disposed on opposing sides (or only one side) of a discontinuity in the bone. Placement of fasteners may be facilitated with a guide device that directs placement of a wire, a drill, and/or a bone screw, among others.

The extension member (and/or a plate member thereof) may be coupled to the bone plate. Coupling may be performed before or after the bone plate is disposed on and/or secured to bone. Coupling may be performed by any suitable coupling mechanism, such as placement of one or more threaded fasteners into threaded engagement with the extension member, bone plate, and/or bone, among others. In some examples, the position of the extension member may be adjusted after coupling and then the extension member fixed in position. In some examples, the extension member may be coupled to the bone plate after a guide device has been removed from the bone plate.

The extension member may be engaged with the bone external and/or internal to the bone. Engagement with bone may be created at any suitable time relative to coupling to the bone plate. In some examples, engagement with bone may be provided by bending or adjusting the position of the extension member in situ, after the extension member has been coupled and/or secured to the bone plate. In some examples, engagement with bone may be provided before or after coupling the extension member to the bone plate by placement of one or more fasteners through an opening of a plate member of the extension and into bone.

The bone plate and/or extension member may be removed at any suitable time. In some examples, the bone plate and extension member may be left in place indefinitely. In some examples, the extension member may be removed selectively and the bone plate left in position for a longer period of time. In some examples, the extension member and bone plate may be removed at a suitable time, such as after sufficient healing has occurred.

### VI. EXAMPLES

The following examples describe selected aspects and embodiments of systems for expanded stabilization of bones, such as a distal portion of a radius bone These selected aspects and embodiments include exemplary plate/extension members, exemplary sets of plate members, and an exemplary guide device that uses the same coupling site on a bone plate as a plate member, among others. These examples are included for illustration and are not intended to limit or define the entire scope of the present teachings.

### Example 1

### Unitary Plate Extension with a Protection

This example describes an exemplary plate extension that is unitary; see FIG. 7.

Plate extension 102 may have a body including a plate member 103 and one or more projections 104, such as a tine 105, extending from an inner surface 106 of the plate member toward bone.

The plate member may include an overlapping region 107 and an extension region 108. The overlapping region and the extension region may be parallel and/or coplanar or may be nonparallel, to form a bent plate member, as shown in the present illustration. The overlapping region may be attached to a generally planar bone plate and/or may be bent, for example, peri-operatively, to fit onto a nonplanar bone plate shaped to fit onto a bone.

### Example 2

### Sets of Plate Components

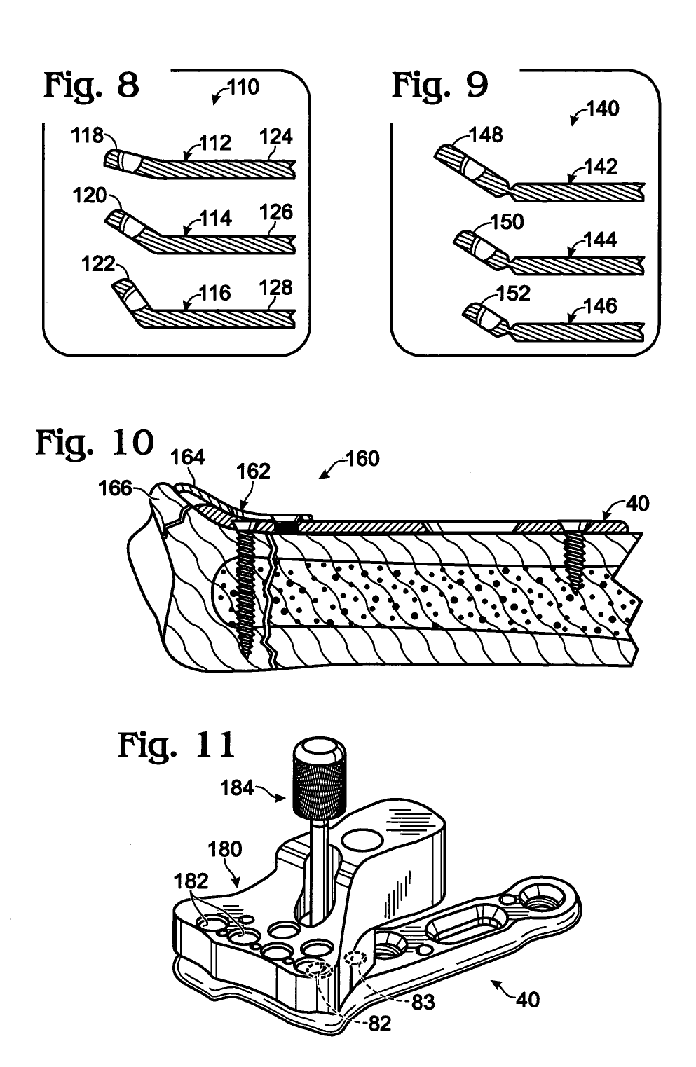

This example describes exemplary sets of plate components (or plate members) that may be included in kits for stabilization of bones, such as the distal radius; see FIGS. 8 and 9.

FIG. 8 shows selected distal regions of an exemplary set 110 of plate components 112, 114, 116 for stabilizing distal fragments of a fractured radius. The plate components may have extension regions 118, 120, 122 disposed at different orientations relative to their respective overlapping regions 124, 126, 128, to define different angles. A surgeon thus may select a suitable plate component from set 110 according to the particular anatomy and/or fracture condition of each individual patient (and/or based on the desired placement of the plate component). The plate components also may be bent pre- and/or peri-operatively, to make adjustments in the orientation of the extension regions. Accordingly, in some examples, the plate components may have a junction region configured to facilitate bending, as described above in relation to FIG. 5. In some examples, the extension regions may define openings with a semi-spherical geometry, to allow placements of fasteners (e.g., with semi-spherical heads) at a range of permitted angles. Alternatively, or in addition, the openings may include an internal thread to lock the fasteners to the extension regions.

FIG. 9 shows selected portions of another exemplary set 140 of plate components 142, 144, 146 for stabilizing distal fragments of a fractured radius. The plate components may have extension regions 148, 150, 152 of different lengths. A surgeon thus may select a suitable component from set 140 according to the particular anatomy and/or fracture condition of each individual patient (and/or based the desired placement of the plate component).

### Example 3

### System for Expanded Bone Stabilization

This example describes another exemplary system for expanded stabilization of bones, such as a radius bone; see FIG. 10.

System 160 may include bone plate 40 and a plate member 162 coupled to the bone plate. The plate member may include an extension region 164 that extends beyond a region of the distal end of the bone plate, to engage a distal fragment 166 of the radius bone. The extension region may rely at least substantially on surface contact between the extension region and the bone to stabilize the distal fragment, rather than using a projection(s) that extends into the distal fragment from the extension region. However, the extension region may include one or more openings (see FIGS. 4 and 5) to allow optional placement of a fastener(s) through the opening(s).

### Example 4

### Guide Device

This example describes an exemplary guide device to assist placement of fasteners through openings of a bone plate and into bone; see FIG. 11.

Guide device 180 may be mounted onto a bone plate, such as bone plate 40, adjacent an outer surface of the bone plate. The guide device may include one or more guide channels 182 that direct placement of a wire, a drill, and/or a fastener through aligned openings of the bone plate, to assist in securing the bone plate to bone. The guide device thus may be coupled to the bone plate before or after the bone is secured to bone. The guide device may include a coupling member 184 that threads into an aperture of the bone plate (e.g., aperture 82; also see FIG. 2), to attach the guide device to the bone plate. The same aperture may be used to attach an extension member to the bone plate after the guide device has been removed.

Further aspects of exemplary guide devices and methods of using the guide devices to direct and assist fastener placement, are described in the following patent applications, which is incorporated herein by reference: U.S. patent application Ser. No. 10/968,850, filed Oct. 18, 2004; and U.S. Provisional Patent Application Ser. No. 60/563,860, filed Apr. 19, 2004.

### Example 5

### Selected Embodiments

This example describes selected embodiments of the present teachings, presented as a series of indexed paragraphs.

1. A kit for expanded stabilization of the distal radius, comprising: (A) a bone plate having an outer surface and defining a perimeter and a plurality of openings configured to receive fasteners that secure the bone plate to a distal surface of a radius bone; and (B) a plurality of plate members, each plate member being configured to be attached to the bone plate so that a region of each plate member extends (1) beyond the perimeter at a different angle relative to the outer surface of the bone plate and/or (2) a different distance than the other plate members.

2. A method of stabilizing a radius bone having a distal fragment created by a fracture of the radius bone, comprising: (A) selecting a bone plate including a plurality of openings and a distal end; (B) securing the bone plate to a distal portion of the radius bone with fasteners received in one or more of the openings; and (C) attaching a plate member to the bone plate such that the plate member overlaps the bone plate and extends beyond a region of the distal end of the bone plate, and at least substantially external to the radius bone, to engage the distal fragment.

3. The method of paragraph 2 or 3, wherein the step of selecting a bone plate includes a step of selecting a bone plate shaped to fit onto a volar surface region of the distal portion of the radius bone.

4. The method of any preceding paragraph, wherein the step of securing fixes at least a pair of fragments of the radius bone created by a first fracture, and wherein the step of attaching stabilizes at least one additional fragment of the radius created by a second fracture extending to a lunate facet region of the radius bone.

5. The method of any preceding paragraph, wherein the step of attaching is performed after the step of securing.

6. The method of any preceding paragraph, wherein the step of attaching includes (1) a step of coupling the plate member to the bone plate in a slidable configuration, (2) a step of sliding the plate member translationally, after the step of coupling the plate member, to adjust a position of the plate member on the bone plate, and (3) a step of fixing the position of the plate member.

7. The method of any preceding paragraph, wherein the plate member defines one or more openings, and wherein the step of attaching the plate member includes a step of placing one or more fasteners into the one or more openings and into the distal fragment.

8. The method of any preceding paragraph, further comprising a step of drilling one or more holes in the distal fragment, wherein the step of attaching a plate member includes a step of attaching an extension member that includes the plate member and one or more projections extending from the plate member, and wherein the step of coupling includes a step of placing the one or more projection into the one or more holes.

9. A method of stabilizing a bone, comprising: (A) selecting a bone plate having a plurality of openings and an aperture; (B) disposing the bone plate on a bone to define a footprint of the bone plate on the bone; (C) coupling a guide device to the bone plate using the aperture; (D) placing fasteners through one or more of the plurality of openings of the bone plate and into the bone along one or more paths defined by the guide device; (E) removing the guide device from the bone plate; and (F) attaching a plate member to the bone plate using the same aperture such that the plate member engages the bone outside the footprint.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A system for expanded stabilization of the distal radius, comprising:
a bone plate shaped to fit onto a portion of a radius bone to define a footprint of the bone plate on the radius bone, the bone plate including a distal end, a row of openings adjacent the distal end for receiving fasteners that secure the bone plate to the radius bone, and an aperture proximal to the row of openings; and
a plate member configured to be attached to the bone plate such that the plate member overlaps the bone plate, extending proximally at least to the aperture and extending distally to a position outside the footprint for stabilization of a distal fragment of the radius bone.

2. The system of claim 1, the bone plate having a central axis that conceptually divides the bone plate into a medial portion and a lateral portion configured to be disposed adjacent corresponding medial and lateral surface regions of the radius bone, wherein the plate member is configured to extend adjacent and distally beyond a region of the distal end defined by the medial portion of the bone plate.

3. The system of claim 1, wherein the plate member has a major portion that overlaps the bone plate and a minor portion that extends beyond the distal end.

4. The system of claim 1, wherein the bone plate has an outer surface, and wherein the plate member is configured to be disposed on the outer surface.

5. The system of claim 1, wherein the plate member has an elongate opening configured to overlap the bone plate when the plate member is attached to the bone plate.

6. The system of claim 1, wherein the plate member includes an inner surface configured to face the radius bone, further comprising one or more projections extending from the inner surface of the plate member.

7. The system of claim 6, wherein the one or more projections are unitary with the plate member.

8. The system of claim 1, wherein the bone plate includes a body portion and a head portion disposed distal to the body portion, and wherein the plate member extends at least halfway from the distal end to the body portion when attached to the bone plate.

9. A system for expanded stabilization of the distal radius, comprising:
a bone plate shaped to fit onto a volar surface of a distal portion of a radius bone and including a distal end and a plurality of openings for receiving fasteners that secure the bone plate to the radius bone; and
a plate member configured to be attached to the bone plate such that a major portion of the plate member overlaps the bone plate and a minor portion extends distally beyond a region of the distal end of the bone plate and at least substantially external to the radius bone for engagement with a distal fragment of the radius bone.

10. The system of claim 9, wherein the bone plate has an outer surface with a nonplanar contour, and wherein the major portion of the plate member has an inner surface configured to abut the outer surface of the bone plate and at least substantially follow a region of the nonplanar contour.

11. The system of claim 9, further comprising at least one projection extending from the body of the plate member into the radius bone.

12. The system of claim 11, wherein the plate member includes at least one opening that does not overlap the bone plate, and wherein the at least one projection is a fastener received in the at least one opening.

13. The system of claim 11, wherein the plate member and the at least one projection are unitary.

14. The system of claim 9, the bone plate having a central portion, wherein the plate member extends proximally to the central portion.

15. A system for expanded stabilization of the distal radius, comprising:
a bone plate shaped to fit onto a distal surface region of a radius bone, the bone plate defining a perimeter and a plurality of openings configured to receive fasteners that secure the bone plate to the radius bone; and
a plate member configured to be coupled slidably to the bone plate such that an adjustable portion of the plate member is disposed outside the perimeter.

16. The system of claim 15, wherein the bone plate defines a distal end, and wherein the plate member is configured to slide so that adjustable portion extends beyond the distal end.

17. The system of claim 15, wherein the bone plate is shaped to fit onto a volar region of the distal surface of the radius bone.

18. The system of claim 15, wherein the plate member defines an elongate aperture that guides sliding movement.

19. The system of claim 15, further comprising an extension member, wherein the extension member includes the plate member and at least one projection extending from the plate member into the radius bone outside the perimeter.

20. The system of claim 15, wherein the adjustable portion of the plate member defines one or more openings configured to receive a fastener.
